(19) United States
(12) Patent Application Publication
(10) Pub. No.: US 2013/0137182 A1
(43) Pub. Date: May 30, 2013

(54) BLOOD PRE-TREATMENT APPARATUS AND PRE-TREATMENT METHOD USING THE SAME

(71) Applicants: Yo Han CHOI, Daejeon (KR); Chan Woo Park, Daejeon (KR); Wan Joong Kim, Goyang-si (KR); Chil Seong Ah, Daejeon (KR); Kwang Hyo Chung, Daejeon (KR); Jong-Heon Yang, Daejeon (KR); Gun Yong Sung, Daejeon (KR)

(72) Inventors: Yo Han CHOI, Daejeon (KR); Chan Woo Park, Daejeon (KR); Wan Joong Kim, Goyang-si (KR); Chil Seong Ah, Daejeon (KR); Kwang Hyo Chung, Daejeon (KR); Jong-Heon Yang, Daejeon (KR); Gun Yong Sung, Daejeon (KR)

(73) Assignee: Electronics and Telecommunications Research Institute, Daejeon (KR)

(21) Appl. No.: 13/544,931

(22) Filed: Jul. 9, 2012

(30) Foreign Application Priority Data
Nov. 24, 2011 (KR) 10-2011-0123605

(51) Int. Cl. G01N 33/48; B01D 21/00

(52) U.S. Cl. 436/63; 422/68.1; 422/527; 422/534

(57) ABSTRACT

Provided is a blood pre-treating apparatus including an injecting part for injecting a blood sample, an albumin-removing part including albumin adsorption beads for removing albumins from the blood sample, and an agglutination part including an agglutination reactant, by which blood corpuscles in the blood sample may be agglutinated to form a hemagglutination reactant.

### CROSS-REFERENCE TO RELATED APPLICATIONS

This U.S. non-provisional patent application claims priority under 35 U.S.C. §119 to Korean Patent Application No. 10-2011-0123605, filed on Nov. 24, 2011, in the Korean Intellectual Property Office, the entire contents of which are hereby incorporated by reference.

### BACKGROUND OF THE INVENTION

Embodiments of the inventive concepts relate to a blood pre-treating apparatus and a pre-treatment method using the same, and in particular, to an apparatus configured to obtain a blood plasma from a whole blood and a pre-treatment method using the same.

Albumin accounts for 50% or more of the total protein in the blood plasma and is a major cause of high blood viscosity. The high blood viscosity results in a technical difficulty in treating blood by a microfluidic chip, and thus, a method of removing the albumin should be developed in order to realize a microfluidic diagnostic chip. In addition, since the albumin may serve as a noise in detecting an infinitesimal biomarker, the removal of the albumin is an important one of blood pre-treating steps.

Meanwhile, to analyze various biomarkers in a blood plasma, a step of removing blood corpuscles accounting for 40% or more of a whole blood should be performed in advance of the analysis. In certain cases, a centrifugal separator may be used to obtain a blood plasma removed with blood corpuscles. In many cases, however, blood plasma without blood corpuscles has been obtained by using a filter made of paper or glass fiber. For example, this method has been used when the centrifugal separator is unusable or the blood plasma should be obtained from a small amount of whole blood. Especially, biochips for point-of-care applications are being realized, for the most part, based on this method.

In the cases of removing blood corpuscles with a filter, a physical structure may be used to make a difference in moving speed between blood corpuscle and blood plasma. This difference in moving speed may be used to separate the blood corpuscle from the blood plasma. In this method, however, even when a large amount of whole blood is used, a small amount of blood plasma can be obtained. Furthermore, it takes a long time (e.g., 10 min or more) to separate the blood plasma completely from the blood corpuscle.

### SUMMARY

Embodiments of the inventive concepts provide a blood pre-treating apparatus configured to obtain blood plasma from a whole blood in a short time.

Other example embodiments of the inventive concept provide a blood pre-treating method capable of obtaining blood plasma from a whole blood in a short time.

According to example embodiments of the inventive concepts, a blood pre-treating apparatus may include an injecting part for injecting a blood sample, an albumin-removing part including albumin adsorption beads for removing albumins from the blood sample, and an agglutination part including an agglutination reactant, by which blood corpuscles in the blood sample may be agglutinated to form a hemagglutination reactant.

In example embodiments, the apparatus may further include a filtering part for removing the hemagglutination reactant from the blood sample.

In example embodiments, the apparatus may further include at least one detecting part configured to detect a biomarker in the blood sample.

In example embodiments, the apparatus may further include a fluid channel provided between the filtering part and the detecting part.

In example embodiments, the albumin-removing part may further include a supporting structure configured to physically support the albumin adsorption beads.

In example embodiments, the apparatus may further include a fluid channel provided between the injecting part and the albumin-removing part.

In example embodiments, the agglutination reactant may be provided to cover an inner surface of the agglutination part.

In example embodiments, the agglutination reactant may be a specific antibody to the blood corpuscle or blood serum including the specific antibody. The specific antibody may be a monoclonal antibody, a polyclonal antibody or a complex of any type of antibody including protein A or protein G.

In example embodiments, the injecting part may include the agglutination reactant.

According to example embodiments of the inventive concepts, a method of pre-treating blood may include removing albumins from a blood sample using albumin adsorption beads, and agglutinating blood corpuscles in the blood sample using an agglutination reactant to form a hemagglutination reactant.

In example embodiments, the method may further include removing the hemagglutination reactant using a filtering device.

In example embodiments, the method may further include detecting at least one biomarker in the blood sample.

In example embodiments, the agglutination reactant may be a specific antibody to the blood corpuscle or blood serum including the specific antibody. The specific antibody may be a monoclonal antibody, a polyclonal antibody or a complex of any type of antibody including protein A or protein G.

### BRIEF DESCRIPTION OF THE DRAWINGS

Example embodiments will be more clearly understood from the following brief description taken in conjunction with the accompanying drawings. The accompanying drawings represent non-limiting, example embodiments as described herein.

FIG. 1 is a schematic plan view illustrating a blood pre-treating apparatus according to example embodiments of the inventive concept;

FIG. 2 is a schematic plan view illustrating an albumin-removing part of a blood pre-treating apparatus according to example embodiments of the inventive concept;

FIG. 3 is an image showing a technical effect according to example embodiments of the inventive concept;

FIG. 4 is a schematic diagram illustrating an agglutination reaction occurring in the agglutination part of the blood pre-treating apparatus according to example embodiments of the inventive concept;

FIGS. 5 and 6 are images exemplarily showing a technical effect that can be achieved by the agglutination part of the blood pre-treating apparatus according to example embodiments of the inventive concept.

It should be noted that these figures are intended to illustrate the general characteristics of methods, structure and/or materials utilized in certain example embodiments and to supplement the written description provided below. These drawings are not, however, to scale and may not precisely reflect the precise structural or performance characteristics of any given embodiment, and should not be interpreted as defining or limiting the range of values or properties encompassed by example embodiments. For example, the relative thicknesses and positioning of molecules, layers, regions and/or structural elements may be reduced or exaggerated for clarity. The use of similar or identical reference numbers in the various drawings is intended to indicate the presence of a similar or identical element or feature.

### DETAILED DESCRIPTION

Example embodiments of the inventive concepts will now be described more fully with reference to the accompanying drawings, in which example embodiments are shown. Example embodiments of the inventive concepts may, however, be embodied in many different forms and should not be construed as being limited to the embodiments set forth herein; rather, these embodiments are provided so that this disclosure will be thorough and complete, and will fully convey the concept of example embodiments to those of ordinary skill in the art. In the drawings, the thicknesses of layers and regions are exaggerated for clarity. Like reference numerals in the drawings denote like elements, and thus their description will be omitted.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Like numbers indicate like elements throughout. As used herein the term “and/or” includes any and all combinations of one or more of the associated listed items. Other words used to describe the relationship between elements or layers should be interpreted in a like fashion (e.g., “between” versus “directly between,” “adjacent” versus “directly adjacent,” “on” versus “directly on”).

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of example embodiments.

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of example embodiments. As used herein, the singular forms “a,” “an” and “the” are intended to include the plural forms as well, unless the context clearly indicates otherwise. It will be further understood that the terms “comprises”, “comprising”, “includes” and/or “including,” if used herein, specify the presence of stated features, integers, steps, operations, elements and/or components, but do not preclude the presence or addition of one or more other features, integers, steps, operations, elements, components and/or groups thereof.

Example embodiments of the inventive concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the inventive concepts should not be construed as limited to the particular shapes of regions illustrated herein but are to include deviations in shapes that result, for example, from manufacturing. For example, an implanted region illustrated as a rectangle may have rounded or curved features and/or a gradient of implant concentration at its edges rather than a binary change from implanted to non-implanted region. Likewise, a buried region formed by implantation may result in some implantation in the region between the buried region and the surface through which the implantation takes place. Thus, the regions illustrated in the figures are schematic in nature and their shapes are not intended to illustrate the actual shape of a region of a device and are not intended to limit the scope of example embodiments.

Unless otherwise defined, all terms (including technical and scientific terms) used herein have the same meaning as commonly understood by one of ordinary skill in the art to which example embodiments of the inventive concepts belong. It will be further understood that terms, such as those defined in commonly-used dictionaries, should be interpreted as having a meaning that is consistent with their meaning in the context of the relevant art and will not be interpreted in an idealized or overly formal sense unless expressly so defined herein.

There are 5×10⁹ red corpuscles and 5×10⁶ or more white corpuscles in 1 ml human blood, and thus, the blood corpuscles account for more than 40% of a blood volume. Various diagnoses on blood may be performed to detect a fluid portion except for the blood corpuscles (i.e., proteins contained in blood plasma), and thus, a step of removing the blood corpuscles may be performed before the blood diagnoses. In large hospitals, a centrifugal separator may be used to obtain the blood plasma removed with the blood corpuscles. In many cases, however, the removal of the blood corpuscles has been performed by using a physical filtering device. For example, the removal of the blood corpuscles may include supplying a blood sample into a blood plasma filter made of paper or glass fiber. In this case, the blood corpuscles may be separated from the blood plasma, due to a difference in a moving speed therebetween caused by a three-dimensional mesh structure. However, much of the blood plasma may remain mixed with the blood corpuscles, thereby deteriorating a recovery rate and requiring a large amount of blood sample. In addition, it takes a long time to separate the blood plasma completely from the blood corpuscle. According to example embodiments of the inventive concept, a method of agglutinating the blood corpuscles forcibly may be used to remove the agglutinated blood corpuscle complex rapidly and improve efficiency in obtaining the blood plasma.

Since the blood plasma contains various kinds of proteins with a high concentration of about 60-80 mg/ml, the blood plasma may exhibit a high viscosity. Here, albumin accounting for 50-70% of the total protein in the blood plasma may serve as a noise or background in detecting an infinitesimal protein. In addition, the high viscosity of the blood plasma may lead to deterioration in flowability of a micro structure for detecting a protein. According to example embodiments of the inventive concept, the albumin may be removed from the blood plasma, and thus, this enables to detect easily an infinitesimal protein, to decrease viscosity of a sample, and to improve the flowability of the micro structure.

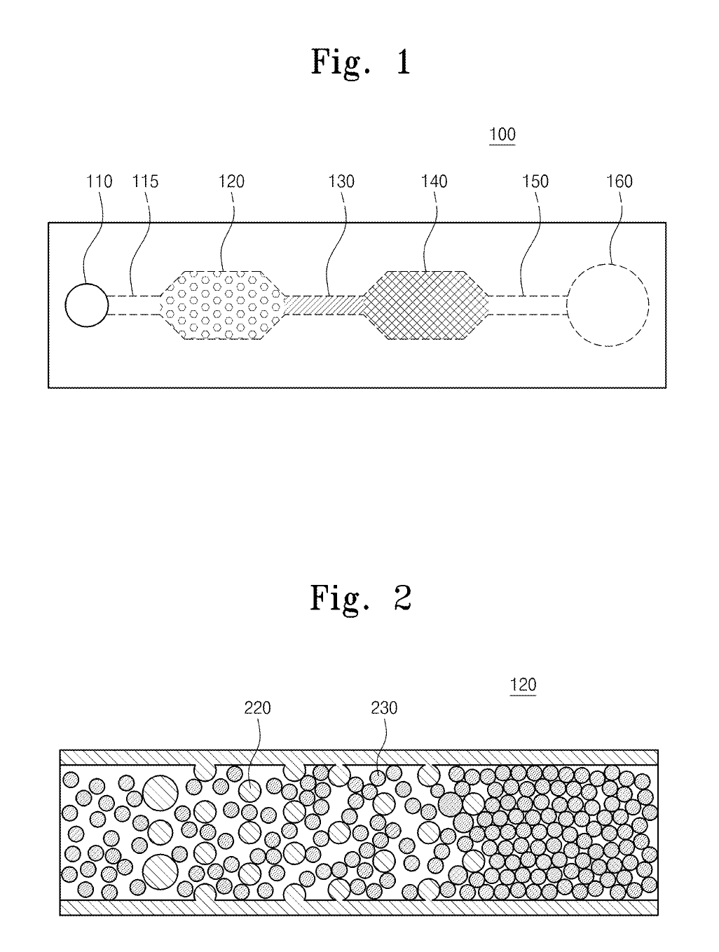

FIG. 1 is a schematic plan view illustrating a blood pre-treating apparatus according to example embodiments of the inventive concept.

Referring to FIG. 1, a blood pre-treating apparatus 100 may include an injecting part 110, in which a blood sample may be injected, an albumin-removing part 120 including albumin adsorption beads (e.g., 230 of FIG. 2), which may be used to remove albumins in the blood sample, and an agglutination part 130 including an agglutination reactant (e.g., 320 of FIG. 4), which may agglutinate blood corpuscles (e.g., 310 of FIG. 4) in the blood sample and form a hemagglutination reactant (e.g., 330 of FIG. 4).

In addition, the blood pre-treating apparatus 100 may include a filtering part 140 to remove the hemagglutination reactant formed in the agglutination part 130, and at least one detecting part 160 to detect a biomarker in the blood sample, in which the albumin and blood corpuscle may be removed. Furthermore, the blood pre-treating apparatus 100 may further include fluid channels 115 and 150 provided between the injecting part 110 and the albumin-removing part 120 and between the filtering part 140 and the detecting part 160.

A blood sample containing the hemagglutination reactant may be flowed through the filtering part 140 provided with a filter. In example embodiments, the filter may be configured to have pores with a pore size much greater than that of a convention filter to be used in a blood plasma separation. As a result, the hemagglutination reactant may be removed, while the remainder of the blood sample may be injected into the detecting part 160.

Although not shown, the injecting part 110 of the blood pre-treating apparatus 100 may be also configured to include the agglutination reactant.

The blood pre-treating apparatus 100 may be configured to be able to perform various operations on the blood sample. For example, the blood sample may be moved, stopped, accelerated, separated, or replaced by the blood pre-treating apparatus 100 or mixed with other fluid (e.g., a test solution)). In example embodiments, the blood pre-treating apparatus 100 may be fabricated in consideration of various variables affecting fluid flow (for example, width, depth, and length of fluid channel, the kind of polymer material, the kind of the fluid, a contact angle, or types and installation positions of pump and valve).

Example embodiments of the inventive concept may not be limited to the depicted example, in which the blood pre-treating apparatus 100 includes one detecting part 160. For example, a plurality of detecting parts 160 may be provided in the blood pre-treating apparatus 100 to detect various biomarkers in a blood sample. In example embodiments, the detecting parts 160 may be sequentially connected in series to the fluid channel 150. In other embodiments, the fluid channel 150 may include a plurality of channels, each of which may be connected to the corresponding one of the detecting parts 160.

The blood pre-treating apparatus 100 and a pre-treating method using the same will be described in more detail below.

FIG. 2 is a schematic plan view illustrating an albumin-removing part of a blood pre-treating apparatus according to example embodiments of the inventive concept.

Referring to FIGS. 1 and 2, when a blood sample is injected into the injecting part 110, the blood sample may be flowed into the albumin-removing part 120 through the fluid channel 115. The albumin-removing part 120 may include the albumin adsorption beads 230 and a supporting structure 220 configured to support and fix physically the albumin adsorption beads 230. The supporting structure 220 may be provided to have a shape with a plurality of pillars. The pillars of the supporting structure 220 may allow the albumin adsorption beads 230 to be fastened to the albumin-removing part 120. The supporting structure 220 may be formed of the same material as a body of the blood pre-treating apparatus 100. For example, the supporting structure 220 may be formed using a process of forming the blood pre-treating apparatus 100. In example embodiments, the supporting structure 220 may be easily and simply formed using a simple method, such as an injection or extrusion molding, during the process of forming the blood pre-treating apparatus 100.

In example embodiments, the albumin adsorption beads 230 may be Reactive Blue2, Cibacron Blue 3G-A, or Cibacron Blue F3FA that can be obtained from Sigma Chemical Company, but the amount and an species of the albumin adsorption beads may not be limited thereto. The albumin protein of the blood sample flowed in the albumin-removing part 120 may be adsorbed on the albumin adsorption beads 230 in several seconds, and the remainder of the blood sample may be flowed out through the albumin-removing part 120.

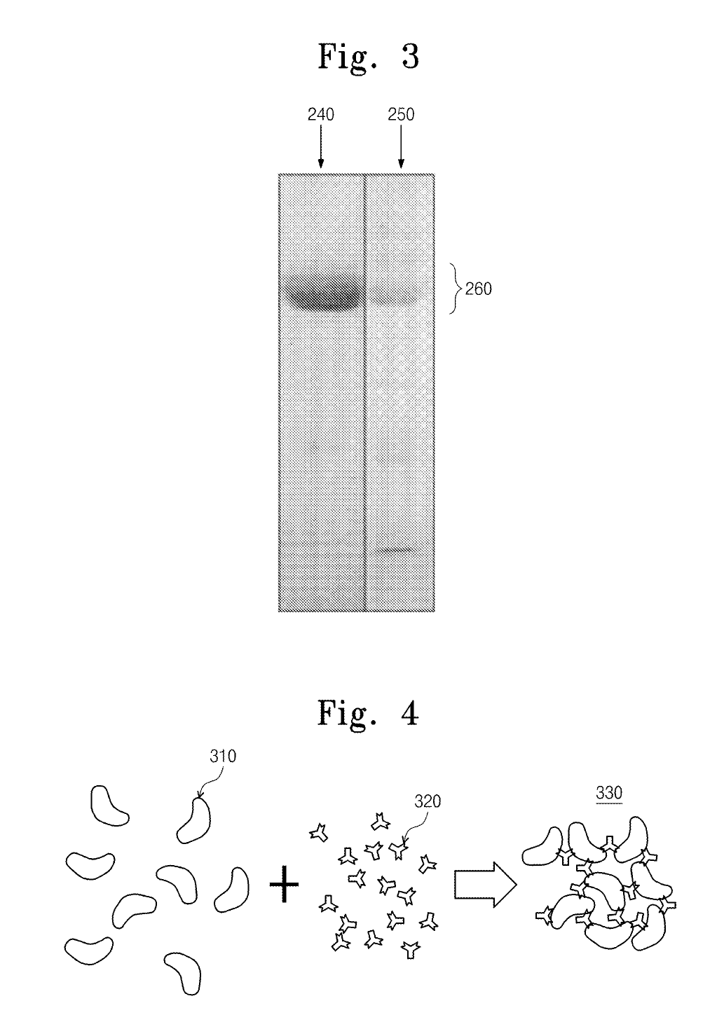

FIG. 3 is an image exemplarily showing a technical effect according to example embodiments of the inventive concept. In detail, FIG. 3 shows an experimental result of an electrophoretic analysis on a sample, to which the albumin-removing part of the blood pre-treating apparatus according to example embodiments of the inventive concept was used.

In FIG. 3, the left image 240 shows a result of Sodium Dodecyl Sulfate PolyAcrylamide Gel Electrophoresis (SDS-PAGE) analysis on a blood sample, to which the removal process using the albumin-removing part 120 was not performed, while the right image 250 shows a result of SDS-PAGE analysis on other blood sample, to which the removal process using the albumin-removing part 120 had been performed. FIG. 3 shows that albumin was smaller in the blood sample 250 than in the blood sample 240. This means that an amount of albumin can be reduced by flowing blood through the albumin adsorption beads 230 of the albumin-removing part 120.

FIG. 4 is a schematic diagram illustrating an agglutination reaction occurring in the agglutination part of the blood pre-treating apparatus according to example embodiments of the inventive concept.

Referring to FIG. 4, the agglutination reactant 320 may be a specific antibody to the blood corpuscle 310 to be removed or blood serum including the specific antibody. For example, about 10⁶ human blood corpuscles may be injected four or more times into an abdominal cavity of a mouse at intervals of three weeks, and after 7-14 days, blood serum may be obtained from a mouse blood. The blood serum may include an antibody specific to the human blood corpuscle. An antibody specific to a blood corpuscle may be prepared using a conventional method, and an animal for the injection of human blood corpuscles may not be limited to the mouse; for example, one of rabbit, goat, horse, and cow may be used as an animal for the injection of human blood corpuscles. The antibody specific to a human blood corpuscle may be separated using an immunoaffinity method according to the purpose of its usage.

The agglutination reactant 320 may be a specific antibody to blood corpuscles in a blood sample or blood serum including the specific antibody. The specific antibody may be a monoclonal antibody, a polyclonal antibody or a complex of any type of antibody including protein A or protein G. The agglutination reactant 320 may be provided to cover an inner surface of the agglutination part 130 of FIG. 1.

Example embodiments of the inventive concepts may not be limited to the afore-described examples, in which the agglutination reactant 320 may be the antibody for agglutinating the blood corpuscles, and for example, the agglutination reactant 320 may be blood serum including other antibody, except for the antibody for agglutinating the blood corpuscles.

If a blood sample is flowed in the agglutination part with the agglutination reactant 320, the agglutination reactant 320 may be mixed with the blood sample. During this process, the agglutination reactant 320 with two junctions may be connected to two blood corpuscles 310, thereby forming the hemagglutination reactant 330.

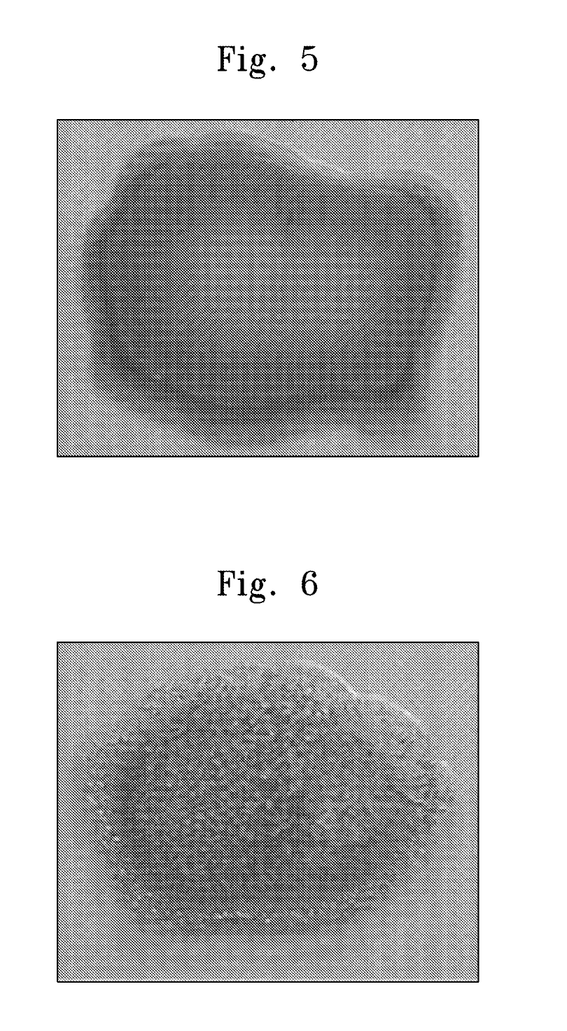

FIGS. 5 and 6 are images exemplarily showing a technical effect that can be achieved by the agglutination part of the blood pre-treating apparatus according to example embodiments of the inventive concept.

In more detail, FIG. 5 is obtained from a human blood sample, to which blood serum of a mouse without immunity to human blood corpuscle was injected, and shows that a blood corpuscle had a uniformly dispersed shape. FIG. 6 is obtained from a human blood sample, to which blood serum of a mouse immunized to human blood corpuscle was injected, and shows that the hemagglutination reactant 330 of FIG. 4 was formed.

In the case where the agglutination reactant according to example embodiments of the inventive concept is used to form a hemagglutination reactant from blood corpuscles in a blood sample, the hemagglutination reactant can be removed from the blood sample by flowing through the filtering part 140 of FIG. 1.

According to example embodiments of the inventive concepts, the blood pre-treating apparatus may be configured to include albumin adsorption beads, which are used to remove albumins from a whole blood in a short time. This enables to obtain blood plasma with low viscosity. As a result, it is possible to perform easily a blood treatment process for detecting an infinitesimal protein, to reduce an amount of blood sample required for the detection, and to reduce the process time required to obtain the blood plasma. In other words, the detection process can be rapidly and exactly performed.

In addition, the agglutination reactant may be used to agglutinate blood corpuscles in a whole blood, thereby facilitating the obtaining of the blood plasma. Accordingly, the blood pre-treating process can be performed with high efficiency in obtaining the blood plasma.

While example embodiments of the inventive concepts have been particularly shown and described, it will be understood by one of ordinary skill in the art that variations in form and detail may be made therein without departing from the spirit and scope of the attached claims.

What is claimed is:

1. A blood pre-treating apparatus, comprising:
an injecting part for injecting a blood sample;
an albumin-removing part including albumin adsorption beads for removing albumins from the blood sample; and
an agglutination part including an agglutination reactant, by which blood corpuscles in the blood sample are agglutinated to form a hemagglutination reactant.

2. The apparatus of claim 1, further comprising, a filtering part for removing the hemagglutination reactant from the blood sample.

3. The apparatus of claim 1, further comprising, at least one detecting part configured to detect a biomarker in the blood sample.

4. The apparatus of claim 3, further comprising, a fluid channel provided between the filtering part and the detecting part.

5. The apparatus of claim 1, wherein the albumin-removing part further comprises a supporting structure configured to physically support the albumin adsorption beads.

6. The apparatus of claim 1, further comprising, a fluid channel provided between the injecting part and the albumin-removing part.

7. The apparatus of claim 1, wherein the agglutination reactant is provided to cover an inner surface of the agglutination part.

8. The apparatus of claim 1, wherein the agglutination reactant is a specific antibody to the blood corpuscle or blood serum including the specific antibody.

9. The apparatus of claim 8, wherein the specific antibody is a monoclonal antibody, a polyclonal antibody or a complex of any type of antibody including protein A or protein G.

10. The apparatus of claim 1, wherein the injecting part comprises the agglutination reactant.

11. A method of pre-treating blood, comprising:
removing albumins from a blood sample using albumin adsorption beads; and
agglutinating blood corpuscles in the blood sample using an agglutination reactant to form a hemagglutination reactant.

12. The method of claim 11, further comprising, removing the hemagglutination reactant using a filtering device.

13. The method of claim 11, further comprising, detecting at least one biomarker in the blood sample.

14. The method of claim 11, wherein the agglutination reactant is a specific antibody to the blood corpuscle or blood serum including the specific antibody.

15. The method of claim 14, wherein the specific antibody is a monoclonal antibody, a polyclonal antibody or a complex of any type of antibody including protein A or protein G.
